(19) United States
(12) Patent Application Publication
(10) Pub. No.: US 2021/0236342 A1
(43) Pub. Date: Aug. 5, 2021

(54) Wound Dressings and Systems with Low-Flow Therapeutic Gas Sources for Topical Wound Therapy and Related Methods

(71) Applicant: KCI Licensing, Inc., San Antonio, TX (US)

(72) Inventors: Justin Alexander Long, Bournemouth, Dorset (GB); Christopher Brian Locke, Bournemouth, Dorset (GB); Timothy Mark Robinson, Blandford Forum (GB)

(21) Appl. No.: 16/757,793

(22) PCT Filed: Oct. 24, 2017
(86) PCT No.: PCT/US2018/057214

§ 371 Date: Apr. 21, 2020

Related U.S. Application Data
(60) Provisional application No. 62/576,487, filed on Oct. 24, 2017.

(51) Int. Cl. A61F 13/02; A61M 1/00

(52) U.S. Cl. CPC A61F 13/0216; A61F 13/022; A61F 13/0223; A61F 2013/00255; A61M 1/94; A61M 2202/0208; A61F 13/0209

(57) ABSTRACT

This disclosure includes wound dressings and systems with low-flow, high-concentration therapeutic gas sources for topical wound therapy and related methods. Some dressings, which are configured to be coupled to tissue to facilitate delivery of therapeutic gas to the tissue, comprise a manifold that defines a plurality of gas passageways, the manifold configured to allow communication of therapeutic gas to the tissue; a sorbent material configured to be disposed above or below the manifold and to capture exudate; and a gas-occlusive layer configured to be disposed over the manifold and the sorbent material and coupled to the tissue such that an interior volume containing the manifold and the sorbent material is defined between the gas-occlusive layer and the tissue and the gas-occlusive layer limits escape of therapeutic gas from the interior volume.

### CROSS-REFERENCE TO RELATED APPLICATIONS

The application claims the benefit of U.S. Provisional Application No. 62/576,487, filed Oct. 24, 2017, the contents of which are incorporated herein in its entirety.

### BACKGROUND

### 1. Field of Invention

The present invention relates generally to wound dressings, and more specifically, but not by way of limitation, to wound dressings and systems with low-flow therapeutic gas sources for topical wound therapy and related methods.

### 2. Description of Related Art

Clinical studies and practice have shown that topical applications of therapeutic gas, such as, for example, oxygen, can improve wound healing, especially in chronic wounds. Topical applications of therapeutic gas can reduce tissue inflammation and/or improve tissue proliferation (e.g., improve collagen synthesis, growth factor production, angiogenesis, and/or the like).

Traditional oxygen-based therapies can supply oxygen at relatively low-flow rates, such as, for example, between 3 and 50 milliliters per hour. Although these traditional oxygen-based therapies can supply high-purity oxygen (e.g., via electrolysis of atmospheric water vapor) to wound dressings, these therapies may require days of operation before the oxygen concentration at the target tissue is greater than 80 percent at least because of ineffective distribution and/or retention of oxygen within the dressing.

Ineffective distribution of oxygen within traditional dressings may be caused at least by empty space within the interior volume defined by the dressing, which allows oxygen to collect without directing the oxygen to the wound, and/or by absorbent material within the dressing that expands as wound exudate fills the dressing, thereby blocking incoming oxygen from reaching target tissue.

Thus, while the clinical benefits of topical applications of therapeutic gas, and in particular, therapeutic oxygen, are known, improvement to the efficacy, convenience, and/or simplicity of therapy systems, components, and related methods may benefit healthcare providers, caregivers, and patients.

### SUMMARY

The present dressings, systems, and/or methods can provide greater efficacy and/or accuracy in the supply and/or delivery of the topical application of therapeutic gas, such as, for example, oxygen, to target tissue. Oxygen, in certain concentrations, can exhibit antimicrobial properties in wound care applications. As such, oxygen can be a more effective antimicrobial than conventional antimicrobials (e.g., copper, silver, zinc, polyhexamethylene biguanide, and/or the like), which may otherwise have potentially negative side effects.

The present dressings, systems, and/or methods can minimize the interior volume defined by the dressing in addition to efficiently distributing therapeutic gas to target tissue and/or providing effective wound exudate management and/or absorbency such that the absorption of exudate does not block or otherwise effectively restrict the permeation of therapeutic gas throughout the dressing and/or uniform application across target tissue. For example, by minimizing the interior volume defined by the dressing, whether by incorporating a high density, low profile manifold material and/or a dense superabsorbent material to fill the interior volume of the dressing and/or by evacuating air within the dressing, using negative pressure, to reduce the interior volume (e.g., prior to the application of therapeutic gas), the present dressings, systems, and/or methods can minimize oxygen stagnation or collection within the interior volume of the dressing and, therefore, direct more oxygen to target tissue. For further example, by providing a dense superabsorbent material within the interior volume of the dressing that swells when the material is exposed to exudate, empty space within the interior volume of the dressing can be reduced when the superabsorbent material is exposed to exudate, thereby further preventing the stagnation of therapeutic gas within the interior volume and forcing the flow of therapeutic gas toward target tissue.

The present dressings, systems, and/or methods can minimize the diffusion of therapeutic gas through the gas-occlusive layer and/or the port of the dressing (e.g., via external sinks and/or leaks), thereby utilizing substantially all of the therapeutic gas provided by the therapeutic gas source.

In these ways and others, the present dressings, systems, and/or methods can increase the efficiency and efficacy of low-flow (e.g., 3 to 50 milliliters per hour), high-concentration (e.g., approximately 99.9 percent) therapeutic gas sources for use in wound therapy.

Some embodiments of the present dressings, which are configured to be coupled to tissue to facilitate delivery of therapeutic gas to the tissue, comprise a manifold that defines a plurality of gas passageways, the manifold configured to allow communication of therapeutic gas to the tissue; an absorbent layer including a foam or a non-woven textile, the absorbent layer configured to be disposed below the manifold and to draw exudate away from the tissue; a patient-interface layer configured to be disposed below the absorbent layer and in contact with the tissue, the patient-interface layer defining a plurality of openings configured to allow communication of therapeutic gas and exudate through the patient-interface layer; and a gas-occlusive layer configured to be disposed over the manifold and coupled to the tissue such that an interior volume containing the manifold is defined between the gas-occlusive layer and the tissue and the gas-occlusive layer limits escape of therapeutic gas from the interior volume.

In some embodiments of the present dressings, the absorbent layer has a plurality of perforations and/or openings configured to allow communication of therapeutic gas to the tissue. In some embodiments of the present dressings, the absorbent layer comprises a hydrophilic material, optionally, a superabsorbent polymer.

Some embodiments of the present dressings comprise a sorbent material configured to be disposed below the gas-occlusive layer and to capture exudate. Some embodiments of the present dressings comprise a sorbent layer that includes the sorbent material. In some embodiments of the present dressings, the sorbent layer has a plurality of perforations; the sorbent layer has one or more openings; and/or the sorbent layer has a textured surface comprising a plurality of grooves. In some embodiments of the present dressings, a planform area of the sorbent layer is at least 5 percent smaller than a planform area of the manifold. In some embodiments of the present dressings, the sorbent layer comprises an absorbent material. In some embodiments of the present dressings, the absorbent material comprises a foam, a non-woven textile, or a superabsorbent polymer. In some embodiments of the present dressings, the sorbent layer comprises an adsorbent material. In some embodiments of the present dressings, the adsorbent material comprises a carbon filter.

In some embodiments of the present dressings, the patient-interface layer comprises a polymer, optionally, silicone, polyethylene, ethylene vinyl acetate, a copolymer thereof, or a blend thereof. In some embodiments of the present dressings, the patient-interface layer includes an adhesive configured to couple the patient-interface layer to the tissue.

Some embodiments of the present dressings, which are configured to be coupled to tissue to facilitate delivery of therapeutic gas to the tissue, comprise a manifold that defines a plurality of gas passageways, the manifold configured to allow communication of therapeutic gas to the tissue; a liquid control layer having a plurality of perforations, the liquid control layer configured to be disposed below the manifold to restrict communication of exudate toward the tissue; a gas-occlusive layer configured to be disposed over the manifold and coupled to the tissue such that an interior volume containing the manifold is defined between the gas-occlusive layer and the tissue and the gas-occlusive layer limits escape of therapeutic gas from the interior volume.

Some embodiments of the present dressings comprise a sorbent material configured to be disposed between the manifold and the liquid control layer and to capture exudate.

Some embodiments of the present dressings, which are configured to be coupled to tissue to facilitate delivery of therapeutic gas to the tissue, comprise a manifold that defines a plurality of gas passageways, the manifold configured to allow communication of therapeutic gas to the tissue; a sorbent material configured to be disposed above or below the manifold and to capture exudate; and a liquid control layer having a plurality of perforations, the liquid control layer configured to be disposed below the sorbent material to restrict communication of exudate toward the tissue; and a gas-occlusive layer configured to be disposed over the manifold and the sorbent material and coupled to the tissue such that an interior volume containing the manifold and the sorbent material is defined between the gas-occlusive layer and the tissue and the gas-occlusive layer limits escape of therapeutic gas from the interior volume.

In some embodiments of the present dressings, the liquid control layer comprises a foam or a non-woven textile. In some embodiments of the present dressings, the liquid control layer comprises a hydrophilic material, optionally, a superabsorbent material. In some embodiments of the present dressings, the liquid control layer comprises a film.

Some embodiments of the present dressings comprises a patient-interface layer configured to be disposed in contact with the tissue, the patient interface layer defining a plurality of openings configured to allow communication of therapeutic gas and exudate through the patient-interface layer.

In some embodiments of the present dressings, the manifold comprises polyethylene, a polyolefin, a polyether, polyurethane, a co-polyester, a copolymer thereof, or a blend thereof. In some embodiments of the present dressings, the manifold comprises a foam or a non-woven textile.

Some embodiments of the present dressings, which are configured to be coupled to tissue to facilitate delivery of therapeutic gas to the tissue, comprise a first manifold layer and a second manifold layer, each defining a plurality of gas passageways, the first and second manifold layers configured to allow communication of therapeutic gas to the tissue; a sorbent material configured to be disposed between the first and second manifold layers and to capture exudate; and a gas-occlusive layer configured to be disposed over the manifold and coupled to the tissue such that an interior volume containing the first and second manifold layers is defined between the gas-occlusive layer and the tissue and the gas-occlusive layer limits escape of therapeutic gas from the interior volume.

In some embodiments of the present dressings, a planform area of the sorbent layer is at least 5 percent smaller than each of a planform area of the first manifold layer and a planform area of the second manifold layer. In some embodiments of the present dressings, the sorbent material comprises an absorbent material. In some embodiments of the present dressings, the sorbent material comprises an adsorbent material.

Some embodiments of the present dressings comprise a liquid control layer having a plurality of perforations, the liquid control layer configured to be disposed below the sorbent material to restrict communication of exudate toward the tissue.

In some embodiments of the present dressings, the patient interface layer includes an adhesive configured to couple the patient-interface layer to the tissue.

In some embodiments of the present dressings, at least one of the first and second manifold layers comprises polyethylene, a polyolefin, a polyether, polyurethane, a co-polyester, a copolymer thereof, or a blend thereof. In some embodiments of the present dressings, at least one of the first and second manifold layers comprises a foam or a non-woven textile.

Some embodiments of the present dressings, which are configured to be coupled to tissue to facilitate delivery of therapeutic gas to the tissue, comprise a manifold that defines a plurality of gas passageways, the manifold configured to allow communication of therapeutic gas to the tissue; and a gas-occlusive layer configured to be disposed over the manifold and coupled to the tissue such that an interior volume containing the manifold is defined between the gas-occlusive layer and the tissue and the gas-occlusive layer limits escape of therapeutic gas from the interior volume.

Some embodiments of the present dressings comprise one or more ports coupled to or defined by the gas-occlusive layer, wherein the one or more ports are configured to permit communication of therapeutic gas through the gas-occlusive layer and into the interior volume. In some embodiments of the present dressings, for at least one of the one or more ports, a filter configured to filter fluid that flows through the port.

In some embodiments of the present dressings, the filter comprises a layer of material that is bonded to an upper surface or a lower surface of the gas-occlusive layer. In some embodiments of the present dressings, the filter comprises polytetrafluoroethylene, a polyester, a polyamide, a copolymer thereof, or a blend thereof.

Some embodiments of the present dressings comprise a valve coupled to the gas-occlusive layer and configured to relieve pressure within the interior volume when pressure within the interior volume exceeds a threshold pressure. In some embodiments of the present dressings, the valve comprises a one-way valve configured to: permit communication of gas out of the interior volume through the valve; and prevent communication of gas into the interior volume through the valve. In some embodiments of the present dressings, the valve comprises a thin film valve or a check valve.

Some embodiments of the present dressings comprise a sensor configured to detect a presence of therapeutic gas within the interior volume. In some embodiments of the present dressings, the sensor is configured to detect a presence of oxygen within the interior volume. In some embodiments of the present dressings, the sensor comprises a material configured to be disposed within the interior volume and to change color in response to a change in oxygen concentration within the interior volume. In some embodiments of the present dressings, the material comprises a pressure-sensitive paint. In some embodiments of the present dressings, material comprises a redox indicator. In some embodiments of the present dressings, the redox indicator comprises methylene blue, phenosafranine, indigo carmine, resazurin, N-phenylanthranilic acid, and/or neutral red. In some embodiments of the present dressings, the material is disposed on a lower surface of the gas-occlusive layer. In some embodiments of the present dressings, the sensor comprises: a layered silicate; a cationic surfactant; an organic colorant; and a reducing agent.

Some embodiments of the present dressings comprise a sensor configured to detect a pH of fluid within the interior volume. In some embodiments of the present dressings, the sensor comprises a material configured to be disposed within the interior volume and to change color in response to a change in pH of fluid within the interior volume. In some embodiments of the present dressings, the material is configured to absorb carbon dioxide. In some embodiments of the present dressings, the material is configured to absorb ammonia. In some embodiments of the present dressings, the material is disposed on a lower surface of the gas-occlusive layer.

In some embodiments of the present dressings, the gas-occlusive layer includes an adhesive configured to couple the gas-occlusive layer to the tissue. In some embodiments of the present dressings, the gas-occlusive layer has a thickness that is between approximately 15 micrometers (μm) and approximately 40 μm. In some embodiments of the present dressings, the gas-occlusive layer comprises polyurethane, polyethylene, polyvinyl acetate, polyvinyl chloride, polyvinylidene chloride, isobutylene, a halogenated isomer, a copolymer thereof, or a blend thereof.

Some embodiments of the present systems include one of the present dressings; an oxygen source; and a conduit configured to be coupled between the oxygen source and the dressing to permit communication of oxygen from the oxygen source into the interior volume of the dressing.

In some embodiments of the present systems, the oxygen source comprises an electrolytic oxygen source. In some embodiments of the present systems, the oxygen source is configured to produce oxygen at a flow rate that is less than approximately 100 milliliters per hour (mL/hour), optionally, less than approximately 50 mL/hour. In some embodiments of the present systems, the conduit includes: an elongated core comprising a foam or a non-woven textile; and a sheath comprising a gas-occlusive film; wherein the sheath is disposed around and extends along at least a majority of a length of the core.

Some embodiments of the present systems include one of the present dressings; a conduit configured to be coupled to the dressing to permit communication of therapeutic gas into the interior volume of the dressing, the conduit comprising: an elongated core comprising a foam or a non-woven textile; and a sheath comprising a gas-occlusive film; wherein the sheath is disposed around and extends along at least a majority of a length of the core. In some embodiments of the present systems, a thickness of the core is less than 10 percent of a width of the core. In some embodiments of the present systems, the sheath comprises polyurethane, polyethylene, polyvinyl acetate, polyvinyl chloride, polyvinylidene chloride, isobutylene, a halogenated isomer, a copolymer thereof, or a blend thereof. In some embodiments of the present systems, the core comprises polyethylene, a polyolefin, a polyether, polyurethane, a co-polyester, a copolymer thereof, or a blend thereof.

Some embodiments of the present methods comprise coupling one of the present dressings to a patient's tissue; and introducing therapeutic gas into the interior volume of the dressing. In some embodiments of the present methods, introducing therapeutic gas into the interior volume of the dressing is performed at a flow rate that is less than approximately 100 mL/hour, optionally, less than approximately 50 mL/hour. In some embodiments of the present methods, introducing therapeutic gas into the interior volume of the dressing comprises introducing oxygen into the interior volume of the dressing. In some embodiments of the present methods, oxygen introduced into the interior volume of the dressing is produced via electrolysis. In some embodiments of the present methods, introducing therapeutic gas into the interior volume of the dressing is performed via a conduit including: an elongated core comprising a foam or a non-woven textile; and a sheath comprising a gas-occlusive film; wherein the sheath is disposed around and extends along at least a majority of a length of the core. In some embodiments of the present methods, a thickness of the core is less than 10 percent of a width of the core. In some embodiments of the present methods, the sheath comprises polyurethane, polyethylene, polyvinyl acetate, polyvinyl chloride, polyvinylidene chloride, isobutylene, a halogenated isomer, a copolymer thereof, or a blend thereof. In some embodiments of the present methods, the core comprises polyethylene, a polyolefin, a polyether, polyurethane, a co-polyester, a copolymer thereof, or a blend thereof. Some embodiments of the present methods comprise, prior to introducing therapeutic gas into the interior volume of the dressing, reducing pressure within the interior volume.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the term “substantially” may be substituted with “within [a percentage] of” what is specified, where the percentage includes 0.1, 1, 5, and 10 percent.

The phrase “and/or” means and or. The phrase “and/or” includes any and all combinations of one or more of the associated listed items. To illustrate, A, B, and/or C includes: A alone, B alone, C alone, a combination of A and B, a combination of A and C, a combination of B and C, or a combination of A, B, and C. In other words, “and/or” operates as an inclusive or.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus that “comprises,” “has,” or “includes” one or more elements possesses those one or more elements, but is not limited to possessing only those elements. Likewise, a method that “comprises,” “has,” or “includes,” one or more steps possesses those one or more steps, but is not limited to possessing only those one or more steps.

Any embodiment of any of the apparatuses, systems, and methods can consist of or consist essentially of—rather than comprise/have/include—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Further, an apparatus that is configured in a certain way is configured in at least that way, but it can also be configured in other ways than those specifically described.

Some details associated with the embodiments are described above, and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures. Figures having schematic views are not drawn to scale.

FIG. 1 is a schematic view of an embodiment of the present systems.

FIG. 2 is an exploded perspective view of a first embodiment of the present wound dressings, suitable for use in some embodiments of the present systems.

FIG. 3 is a top view of the dressing of FIG. 2.

FIG. 4A is a cross-sectional side view of the dressing of FIG. 2, taken along line 4A-4A of FIG. 3, shown with a port extending through a portion thereof.

FIG. 4B is a second cross-sectional side view of the dressing of FIG. 2, taken along line 4A-4A of FIG. 3, shown with a port extending through a portion thereof.

FIG. 5 is a top view of an embodiment of a sorbent layer, suitable for use in some embodiments of the present dressings.

FIG. 6 is a top view of an embodiment of a manifold, suitable for use in some embodiments of the present dressings.

FIG. 7 is a cross-sectional end view of an embodiment of a conduit, suitable for use in some embodiments of the present dressings, taken along line 7-7 of FIG. 2.

FIG. 8A is a top view of an embodiment of a manifold, suitable for use in some embodiments of the present dressings.

FIG. 8B is a side view of the manifold of FIG. 8A.

FIG. 9 is an exploded perspective view of a second embodiment of the present wound dressings, suitable for use in some embodiments of the present systems.

FIGS. 10 and 11 depict oxygen concentration data, over time, within dressings having the present gas-occlusive layer.

### DETAILED DESCRIPTION

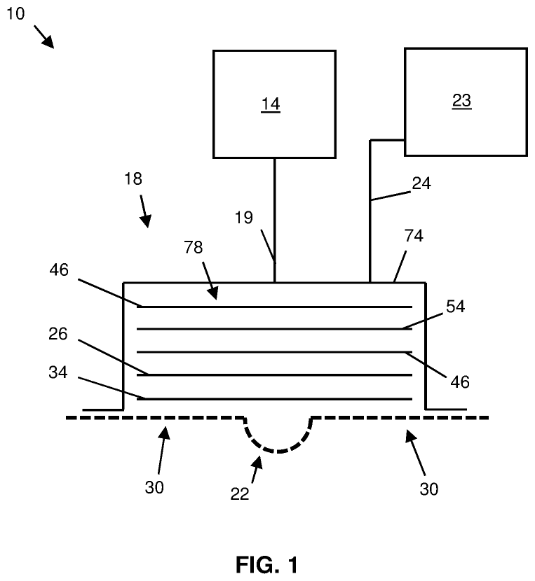

Referring to FIG. 1, shown therein and designated by the reference numeral 10 is one embodiment of the present systems for providing topical wound therapy. System 10 includes a therapeutic gas source 14 and a wound dressing 18 configured to be coupled to target tissue 22 and/or to tissue 30 surrounding the target tissue to facilitate delivery of therapeutic gas to the target tissue.

The term “target tissue” as used herein can broadly refer to a wound (e.g., open or closed), a tissue disorder, and/or the like located on or within tissue, such as, for example, bone tissue, adipose tissue, muscle tissue, neural tissue, dermal tissue, vascular tissue, connective tissue, cartilage, tendons, ligaments, and/or the like. The term “target tissue” as used herein can also refer to areas of tissue that are not necessarily wounded or exhibit a disorder, but include tissue that would benefit from tissue generation. The term “wound” as used herein can refer to a chronic, subacute, acute, traumatic, and/or dehisced incision, laceration, puncture, avulsion, and/or the like, a partial-thickness and/or full thickness burn, an ulcer (e.g., diabetic, pressure, venous, and/or the like), flap, and/or graft.

Therapeutic gas source 14 can be configured to be in fluid communication with dressing 18 via a conduit 19 (as described in further detail below). Therapeutic gas source 14 can be configured to supply therapeutic gas to an interior volume (e.g., 78) defined by dressing 18 (as described in further detail below).

Therapeutic gas source 14 can be configured to supply therapeutic gas to dressing 18 at a “low flow,” which is a volumetric flow rate less than approximately 100 milliliters per hour (mL/hour), such as, for example, less than approximately any one of, or between approximately any two the following: 100, 90, 80, 70, 60, 50, 40, 30, 20, 10, 5, 4, 3, 2, or 1 mL/hour. Therapeutic gas source 14 can be configured to supply any gas, such as, for example, oxygen, that is suitable for treating target tissue 22. Therapeutic gas supplied by therapeutic gas source 14 can comprise a high oxygen concentration, such as an oxygen concentration of at least 80 percent (e.g., 80, 85, 90, 92, 94, 96, 98, 99, 99.9, 99.99 or more percent).

Therapeutic gas source 14 can comprise any suitable device configured to supply therapeutic gas to dressing 18 at one or more of the volumetric flow rates and/or oxygen concentrations described herein, such as, for example, an electrolytic oxygen source (e.g., an oxygen generator), a liquid oxygen reservoir, a reservoir having compressed oxygen gas, and/or the like.

System 10 can include a negative pressure source 23 configured to be in fluid communication with dressing 18 via a conduit 24. In some embodiments, a therapeutic gas source (e.g., 14) and a negative pressure source (e.g., 23) can be configured to be in fluid communication with a dressing (e.g., 18) via a single conduit (e.g., 19) and flow between the therapeutic gas source, the negative pressure source, and the dressing can be controlled by a valve. Negative pressure source 23 can be configured to provide negative pressure within an interior volume (e.g., 78) of dressing 18 such that the volume of the interior volume is reduced and/or negative pressure is applied to target tissue 22 and/or tissue 30 surrounding the target tissue to improve wound healing and/or sealing between the dressing and tissue surrounding the target tissue. As used herein, “negative pressure” can refer to a pressure that is less than a local ambient pressure, such as less than atmospheric pressure. In this way and others, negative pressure source 23 can facilitate the reduction of the volume of an interior volume (e.g., 78) such that the dressing exhibits a low profile and follows the contours of the tissue surrounding the target tissue. To this end, negative pressure source 23 may be configured to provide a negative pressure that is less than or equal to a negative pressure applied to target tissue 22 to trigger therapeutic effects. For example, negative pressure source 23 may be configured to provide a negative pressure ranging from 0.01 to 50 mmHg.

Negative pressure source 23 can comprise a reservoir of gas held within the reservoir at a negative pressure, the gas being in selective communication with therapeutic gas source 14 to provide negative pressure. Negative pressure source 23 can comprise a mechanically and/or electrically-powered device, such as, for example, a vacuum pump, a suction pump, a wall suction port, a micro-pump, and/or the like that can provide negative pressure to therapeutic gas source 14.

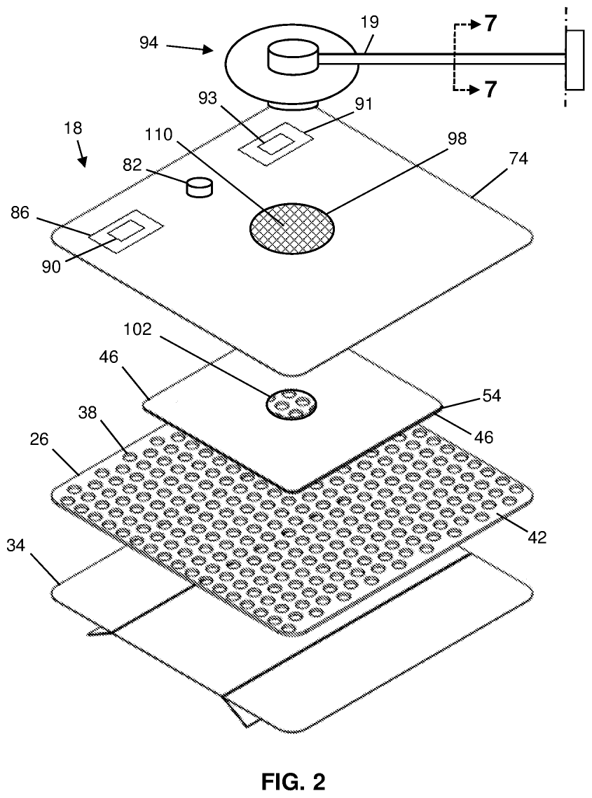

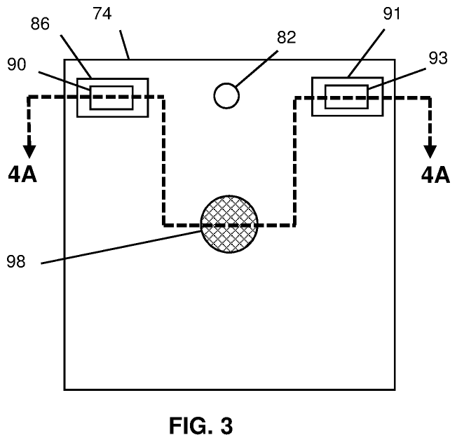

Referring now to FIGS. 1 and 2, dressing 18 can include a patient-interface layer 26 configured to be in contact with target tissue 22 and/or tissue 30 surrounding the target tissue. For example, patient-interface layer 26 may be disposed over target tissue 22 and be in contact with tissue 30 surrounding the target tissue. For further example, patient-interface layer 26 may be disposed over target tissue 22 such that the patient-interface layer fills at least a portion of a recess defined by the target tissue. Patient-interface layer 26 can comprise any suitable planform shape, planform area, thickness, and/or the like that is appropriate to treat target tissue 22.

Patient-interface layer 26 can comprise an adhesive configured to couple the patient-interface layer to target tissue 22 and/or tissue 30 surrounding the target tissue. Such an adhesive can be configured to have low tack properties to minimize patient discomfort and/or tissue trauma as a result of the application, repositioning, and/or removal of patient-interface layer 26 from target tissue 22 and/or tissue 30 surrounding the target tissue. Such an adhesive may comprise any suitable adhesive, such as, for example, an acrylic adhesive, polyurethane gel adhesive, silicone adhesive, hydrogel adhesive, hydrocolloid adhesive, a combination thereof, and/or the like. For example, such an adhesive may be disposed about the edges of a tissue-facing surface of patient-interface layer 26 (i.e., in an arrangement referred to as a “window pane”). Dressing 18 may include a protective liner 34 configured to be disposed on a surface of patient-interface layer 26 such that the protective liner at least partially covers the adhesive (e.g., prior to application of the dressing onto tissue).

Patient-interface layer 26 can comprise a plurality of openings 38 configured to allow communication of therapeutic gas and exudate through the patient-interface layer and/or to promote granulation of target tissue 22. As shown, each of openings 38 of patient-interface layer 26 includes a circular shape. Openings 38 of patient-interface layer 26 can comprise any suitable shape, such as, for example, circular, elliptical, or otherwise round, square, rectangular, hexagonal, or otherwise polygonal. Each of openings 38 of patient-interface layer 26 may be substantially equal in size (e.g., as measured by a maximum transverse dimension of the opening), such as, for example, approximately any one of, or between approximately any two of, the following: 0.1, 0.2, 0.3, 0.4, 0.5, 0.75, 1.0, 1.25, and 1.5 centimeters (cm). In some embodiments, a patient-interface layer (e.g., 26) may comprise openings (e.g., 38) having different sizes.

Patient-interface layer 26 can comprise a plurality of gas passageways 42 defined by any suitable material, such as, for example, an open-cell foam (e.g., reticulated foam). Each gas passageway 42 can comprise a maximum transverse dimension of 400 and 600 micrometers. Patient-interface layer 26 can be hydrophilic. For example, patient-interface layer 26 can be configured to wick away (e.g., by capillary flow through gas passageways 42) exudate from target tissue 22 and/or tissue 30 surrounding the target tissue.

Patient-interface layer 26 can comprise any suitable material, such as, for example, a polymer, optionally, silicone, a hydrogel, polyvinyl alcohol, polyethylene, a polyurethane, polyether, ethylene vinyl acetate, a copolymer thereof, or a blend thereof. In some embodiments, a patient-interface layer (e.g., 26) can serve as or include a scaffold to promote tissue generation. Such a scaffold may comprise any suitable scaffold for soft tissue healing, such as, for example, autograft tissue, collagen, polylactic acid (PLA), polyglycolic acid (PGA), and/or the like. In some embodiments, a patient-interface layer (e.g., 26) may comprise a biodegradable material, such as, for example, PLA, PGA, a polycarbonate, polypropylene fumarate, polycaprolactone, a polymeric blend thereof, and/or the like.

Non-limiting examples of patient-interface layer 26 include Silbione® HC2 products, which are commercially available from Bluestar Silicones International, of Lyon, France, Nanova™ Dressing Perforated Silicone Wound Contact Layers, which are commercially available from Kinetic Concepts, Inc., of San Antonio, Tex., USA, and Bioflex® Performance Materials, which are commercially available from Scapa Healthcare of Windsor, Conn., USA.

Dressing 18 can include one or more manifolds 46. Each manifold 46 can be configured to allow communication of therapeutic gas to target tissue 22 and/or allow communication of exudate to a sorbent material (e.g., 58) (as discussed in further detail below). Manifold 46 may be porous. For example, each manifold 46 can define a plurality of gas passageways 50 to distribute therapeutic gas across the manifold and/or to collect exudate from target tissue 22 across the manifold. Plurality of gas passageways 50 of each manifold 46 can be interconnected to improve distribution and/or collection of fluids across the manifold. For example, gas passageways 50 can be defined by an open-cell foam (e.g., reticulated foam), tissue paper, gauze, a non-woven textile (e.g., felt), and/or the like. In embodiments where manifold 46 comprises a non-woven textile, dressing 18 can comprise two or more manifolds 46 (e.g., one or more on opposing sides of sorbent layer 54). Beneficially, when the volume of interior volume 78 is reduced or minimized, gas passageways 50 of manifolds 46 can continue to distribute therapeutic gas across the manifold and/or to collect exudate from target tissue 22 across the manifold. In this way and others, dressing 18 can minimize the volume within interior volume 78 without affecting the efficacy of the distribution of therapeutic gas to and/or to the collection of exudate from target tissue 22.

Manifold 46 can comprise any suitable material, such as, for example, polyethylene, a polyolefin, a polyether, polyurethane, a co-polyester, a copolymer thereof, or a blend thereof. For example, in embodiments where manifold 46 comprises a foam, such a foam may be polyether-based polyurethane foam. Manifold 46 can comprise any suitable planform shape, planform area, thickness, and/or the like that is appropriate to treat target tissue 22. In embodiments where manifold 46 comprises a non-woven textile, such a non-woven textile can comprise a density ranging from approximately 80 to 150 grams per square meter (GSM) and a thickness ranging from approximately 2 millimeters (mm) to 12 mm. In embodiments where manifold 46 comprises a foam, such a foam can comprise a porosity ranging from approximately 20 to 120 ports per inch (ppi), such as, for example, 45 ppi, and a thickness ranging from approximately 2 mm to 12 mm, such as, for example, 6 mm.

Non-limiting examples of manifold 46 include MEDISPONGE® Foams, which are commercially available from Essentra PLC of Milton Keynes, England, and Exudate Management Systems, which are commercially available from TWE Group GmbH, of Emsdetten, Germany.

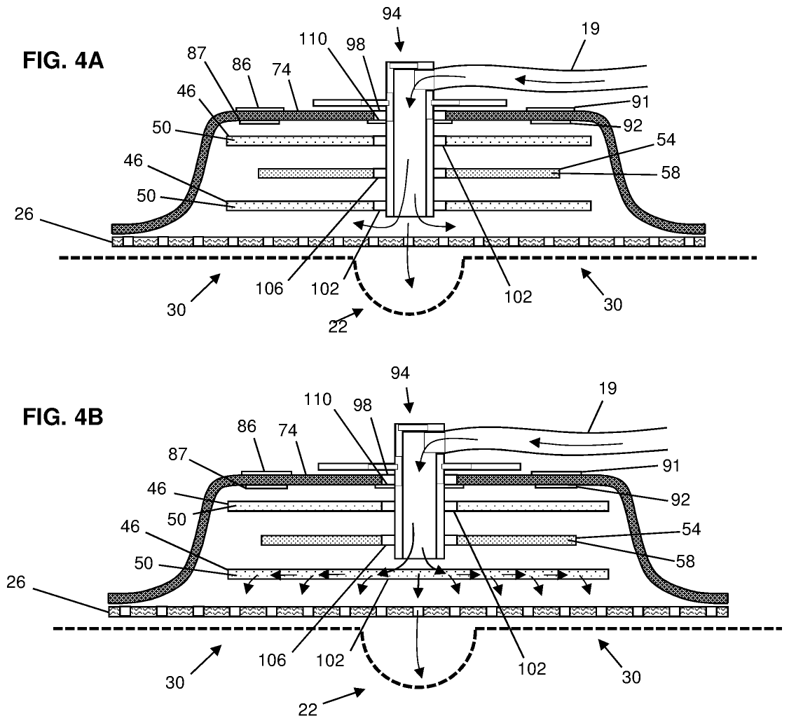

Dressing 18 can include a sorbent layer 54. As shown in FIGS. 2, 4A, and 4B, patient-interface layer 26 can be configured to be disposed below sorbent layer 54. Sorbent layer 54 can include a sorbent material 58 configured to draw exudate away from target tissue 22 and/or tissue 30 surrounding the target tissue. Sorbent material 58 can be disposed below or above one of manifolds 46 to capture exudate. As shown in FIGS. 2, 4A, and 4B, sorbent material 58 can be disposed between a first one of manifolds 46 and a second one of the manifolds. Sorbent layer 54, and, more particularly, sorbent material 58, can comprise any suitable adsorbent or absorbent material. Sorbent layer 54 having absorbent material may comprise a hydrophilic material. Suitable examples of an absorbent material (e.g., a material that tends to swell, by 50 percent or more, due to the binding of liquid within the material) includes a foam, a non-woven textile, a superabsorbent polymer, and/or the like. For example, sorbent material 58 having absorbent material may comprise sodium carboxymethyl cellulose (NaCMC) fiber, alginate fiber, and/or the like. Suitable examples of an adsorbent material (e.g., a material that has a surface onto which liquid binds such that the material does not swell) include carbon filters, such as, for example, an activated charcoal filter and/or the like. Such an activated charcoal filter can be configured to remove nitrogen from therapeutic gas supplied from therapeutic gas source 14 into dressing 18. In this way and others, sorbent material 58 can facilitate the filtration of nitrogen within an interior volume (e.g., 78) of dressing 18.

Non-limiting examples of sorbent material 58 include superabsorbent wound care laminates having a density of 300 grams per square meter (GSM), which are commercially available from Gelok International of Dunbridge, Ohio, USA, and Absorflex™, which has a density of 800 GSM and is commercially available from Texsus S.p.A. of Chiesina Uzzanese, Italy.

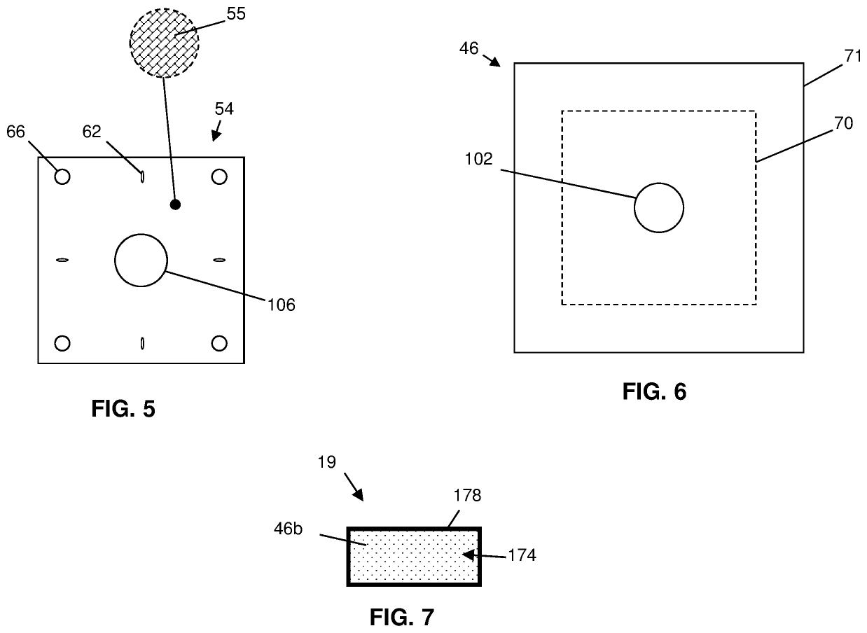

As shown in FIG. 5, sorbent layer 54 can comprise a plurality of perforations 62 and/or a plurality of openings 66, one or more of which are configured to allow fluid communication through the sorbent layer, for example, in instances where sorbent material 58 exhibits gel-blocking. Gel-blocking can occur when sorbent material 58 forms a gel in response to absorption of liquid. Gel-blocking can cause sorbent material 58 to block liquid and/or gas flow through the sorbent material. As shown in FIG. 5, sorbent layer 54 can comprise a textured surface having a plurality of grooves 55 configured to distribute liquid into and/or around sorbent material 58.

In this embodiment, each opening 66 may define an aperture comprising a perimeter that does not substantially change (e.g., does not change by more than 5 percent) in response to fluid flow through the opening. Each perforation 62 may define an aperture comprising a perimeter that substantially changes (e.g., changes by more than 5 percent) in response to fluid flow through the perforation. For example, one or more of perforations 62 may be defined by a slit in sorbent layer 54. Each of openings 66 of sorbent layer 54 may be substantially equal in size (e.g., as measured by a maximum transverse dimension of the opening), such as, for example, approximately any one of, or between approximately any two of, the following: 0.5, 0.75, 1.0, 1.25, and 1.5 cm. Each of perforations 62 of sorbent layer 54 may comprise a size (e.g., as measured by a maximum transverse dimension of the perforation) that is substantially smaller than the size of one or more of openings 66, such as, for example, 50, 60, 70, 80, or 90 percent smaller in size.

Sorbent layer 54 can comprise any suitable planform shape, planform area, thickness, and/or the like appropriate to treat target tissue 22. As shown in FIG. 6, a planform area of sorbent layer 54 (depicted by dotted line 70) is smaller than a planform area of one or more manifolds 46 (depicted by solid line 71) such that, when sorbent layer 54 is disposed between manifolds 46 (i.e., when a manifold is disposed on opposing sides of the sorbent layer), the opposing manifolds can be coupled around a peripheral edge of the sorbent layer to define a pocket. For example, the planform area of sorbent layer 54 can be at least 5 percent smaller, such as, for example, 5, 10, 15, 20, 25, 30, 35, 40, or 45 percent smaller than the planform area of one or more manifolds 46. In this way and others, therapeutic gas can circumvent sorbent layer 54 around its periphery and be distributed from a manifold 46 on a first side of the sorbent layer to a manifold 46 on an opposing second side of the sorbent layer.

Dressing 18 can include a gas-occlusive layer 74. Gas-occlusive layer 74 can be configured to be disposed over one or more manifolds 46 and coupled to tissue 30 surrounding target tissue 22 such that an interior volume 78 containing the manifold(s) is defined between the gas-occlusive layer and the target tissue and such that the gas-occlusive layer limits the escape of therapeutic gas and/or exudate from the interior volume between the gas-occlusive layer and the tissue surrounding the target tissue. Gas-occlusive layer 74 can limit escape of therapeutic gas between the gas-occlusive layer and tissue 30 surrounding target tissue 22 such that, by providing therapeutic gas to dressing 18 at one or more of the volumetric flow rates and/or oxygen concentrations described herein, system 10 can attain an oxygen concentration of at least 80 percent (e.g., 80, 85, 90, 92, 94, 96, 98 or more percent) within interior volume 78 of the dressing within a time duration less than 48 hours, such as, for example, approximately 4 to 8 hours (e.g., approximately any one of, or between approximately any two of: 4, 4.5, 5, 5.5, 6, 6.5, 7, 7.5, and 8 hours). The duration of time required for a dressing (e.g., 18) to attain an oxygen concentration of at least 80 percent may be dependent on one or more dimensions of the dressing and/or the volumetric flow rate of a therapeutic gas source (e.g., 14). For example, for a therapeutic gas source (e.g., 14) that supplies oxygen of approximately 80 percent purity at a volumetric flow rate of approximately 15 mL/hour to a dressing (e.g., 18) that has an interior volume (e.g., 78) spanning 4 inches by 5 inches (e.g., from a plan view), the dressing would attain at least 80 percent pure oxygen within the interior volume in approximately 3 to 3.5 hours.

As shown in FIG. 4A, gas-occlusive layer 74 can be configured to be disposed over sorbent layer 54 such that interior volume 78 contains the sorbent material. In other words, sorbent layer 54, and thus, sorbent material 58, can be configured to be disposed below gas-occlusive layer 74. A portion of gas-occlusive layer 74 can be coupled to tissue 30 surrounding target tissue 22 via patient-interface layer 26. To illustrate, a tissue-facing surface of gas-occlusive layer 74 can comprise an adhesive, such as, for example, an acrylic adhesive, polyurethane gel adhesive, silicone adhesive, a combination thereof, and/or the like, configured to couple the gas-occlusive layer to patient-interface layer 26 and/or tissue 30 surrounding target tissue 22. For example, when gas-occlusive layer 74 is coupled to patient-interface layer 26, such an adhesive may flow through one or more of openings 38 of the patient-interface layer to adhere gas-occlusive layer 74 to tissue 30 surrounding target tissue 22.

Gas-occlusive layer 74 can be sterile such that the gas-occlusive layer provides a viral and/or bacterial barrier to target tissue 22. Gas-occlusive layer 74 can be configured to provide a layer of protection from physical trauma to target tissue 22. In some embodiments, a portion of a gas-occlusive layer (e.g., 74) may be configured to be gas-permeable to provide a suitable (e.g., moist) wound healing environment and/or to prevent passive permeation of therapeutic gas molecules through the gas-occlusive layer. Gas-occlusive layer 74 can comprise an oxygen permeability coefficient (P×10¹⁰), at 25 degrees Celsius, ranging from 0.0003 to 0.5 (e.g., approximately any one of, or between approximately any two of the following: 0.0003, 0.0004, 0.0005, 0.0006, 0.0007, 0.0008, 0.0009, 0.001, 0.005, 0.01, 0.05, 0.1, 0.15, 0.2, 0.25, 0.3, 0.35, 0.4, 0.45, and 0.5), where P is measured in units of [(cm³)(cm)]/[(cm²)(s)(cm Hg)] which represents [(amount of permeate)(gas-occlusive layer thickness)]/[(surface area)(time)(pressure-drop across the gas-occlusive layer)]. Gas-occlusive layer 74 can comprise a moisture vapor transmission rate (MVTR) of at least 250 grams per meters squared per day (g/m²/day). In embodiments where a tissue-facing surface of gas-occlusive layer 74 comprises an adhesive (as discussed above), the adhesive may affect the gas permeability and/or the MVTR of the gas-occlusive layer. To illustrate, for a gas-occlusive layer (e.g., 74) having a film with a thickness of 0.025 mm and an adhesive with a thickness of 0.025 mm, the gas permeability and MVTR of the gas-occlusive layer are half as much as the gas permeability and MVTR of the same gas-occlusive layer without the adhesive.

Gas-occlusive layer 74 may comprise a flexible film, such as, for example, a hydrocolloid sheet. Gas-occlusive layer 74 can comprise any suitable material that limits escape of therapeutic gas and/or exudate through the gas-occlusive layer, such as, for example, polyurethane, polyethylene, polyvinyl acetate, polyvinyl chloride, polyvinylidene chloride, isobutylene, a halogenated isomer (e.g., chlorobutyl and/or bromobutyl), epichlorohydrin, a copolymer thereof, or a blend thereof. Gas-occlusive layer 74 can comprise any suitable planform shape, planform area, thickness, and/or the like that is appropriate to treat target tissue 22. For example, gas-occlusive layer 74 can comprise a thickness that is approximately any one of, or between approximately any two of the following: 5, 10, 15, 20, 25, 30, 35, 40, 45, 50, 55, and 60 micrometers.

Dressing 18 can comprise a valve 82 coupled to gas-occlusive layer 74. Valve 82 can be configured to permit communication of gas out of interior volume 78 through the valve and prevent communication of gas into the interior volume through the valve. For example, valve 82 can be configured to relieve pressure within interior volume 78 when the pressure within the interior volume exceeds a threshold pressure. Such a threshold pressure may range from 8 to 24 mmHg (e.g., approximately any one of, or between approximately any two of the following: 8, 10, 12, 14, 16, 18, 20, 22, and 24 mmHg). Valve 82 can comprise any suitable one-way valve, such as, for example, a ball-check valve, a thin film valve, a diaphragm check valve, and/or the like. In this way and others, valve 82 can be configured to ensure that interior volume 78 does not become over-pressurized with therapeutic gas such that dressing 18 and tissue 30 surrounding target tissue 22 separate to allow therapeutic gas therebetween.

Dressing 18 may comprise one or more sensors 86 configured to collect data indicative of the presence, volume, and/or concentration of therapeutic gas (e.g., oxygen) and/or liquid (e.g., exudate) within interior volume 78. Sensor 86 may operate passively (i.e., the sensor may not require an external power source). Sensor 86 can comprise a material 87 configured to be disposed within interior volume 78. For example, material 87 of sensor 86 can be disposed on a lower (i.e., tissue-facing) surface of gas-occlusive layer 74. Material 87 can be configured to change color in response to a change in concentration of therapeutic gas within the interior volume. For example, material 87 of sensor 86 can comprise a pressure-sensitive paint, a redox indicator (e.g., comprising methylene blue, phenosafranine, indigo carmine, resazurin, N-phenylanthranilic acid, and/or neutral red). In some embodiments, a sensor (e.g., 86) can comprise a layered silicate, a cationic surfactant, an organic colorant, and a reducing agent. For example, in embodiments where material 87 comprises an organic colorant, the colorant can be configured to exhibit a first color when the material is exposed to oxygen having a concentration that is less than or equal to 20 percent. Such an organic colorant can be configured to begin gradually changing color from the first color to a second color when the concentration of oxygen within interior volume 78 becomes greater than approximately 20 percent. Such an organic colorant can be configured to continue gradually changing color from the first color to the second color until the concentration of oxygen within interior volume 78 is approximately 90 to 95 percent, at which time the colorant exhibits only the second color. Sensor 86 may comprise a display 90 configured to indicate, such as, for example, via a color change, the presence, volume, and/or concentration of oxygen within interior volume 78. A non-limiting example of sensor 86 includes the Ageless Eye™ Oxygen Indicator, which is commercially available from Mitsubishi Gas Chemical Company, Inc., of Tokyo, Japan, that is modified to change color in response to oxygen exceeding 20 percent purity.

Dressing 18 may comprise a sensor 91 configured to detect a pH of fluid within interior volume 78. Sensor 91 can comprise a material 92 configured to be disposed within interior volume 78. Similar to material 87, material 92 can be configured to be disposed on a lower (e.g., tissue-facing) surface of gas-occlusive layer 74.

Material 92 can be configured to change color in response to a change in pH of fluid (e.g., liquid, such as, for example, exudate and/or blood) within the interior volume. For example, material 92 of sensor 91 can be configured to absorb carbon dioxide and/or ammonia. In some embodiments, material 92 comprises litmus paper. Like sensor 86, sensor 91 may comprise a display 93 configured to indicate, such as, for example, via a color change, a change in pH of fluid within interior volume 78. By detecting a pH of fluid within interior volume 78, sensor 91 can provide an indication of whether target tissue 22 is exhibiting more or less chronic characteristics at least because chronic wounds are more alkaline than acute wounds. While sensor 86 and sensor 91 are described as distinct sensors, in some embodiments, sensor 86 and sensor 91 may be the same sensor.

Gas-occlusive layer 74 can comprise one or more openings 98 configured to allow communication of therapeutic gas into interior volume 78 of dressing 18. For example, opening 98 of gas-occlusive layer 74 can be configured to receive a port (e.g., 94).

Dressing 18 may comprise one or more ports 94, each of which are configured to be coupled to a respective opening 98 of gas-occlusive layer 74 or defined by the gas-occlusive layer. Port 94 can be configured to allow fluid communication of therapeutic gas from therapeutic gas source 14, through gas-occlusive layer 74, and into interior volume 78 of dressing 18. Port 94 can comprise a filter (e.g., 110) such that the filter filters fluid that flows through the port.

Port 94 can comprise one or more latching and/or interlocking features such that the port can be releasably coupled to therapeutic gas source 14 via conduit 19. For example, port 94 can be configured to be releasably coupled to therapeutic gas source 14 such that the therapeutic gas source can be decoupled from the port without removing dressing 18 from target tissue 22 and/or tissue 30 surrounding the target tissue. Port 94 may comprise an adhesive configured to seal around opening 98 of gas-occlusive layer 74 in order to minimize the diffusion of therapeutic gas between the port and the gas-occlusive layer.

A non-limiting example of port 94 includes the T.R.A.C.™ Pad, which is commercially available from Kinetic Concepts, Inc., of San Antonio, Tex., USA.

As shown in FIGS. 4A and 4B, dressing 18 can be configured such that port 94 can extend through one or more components (e.g., 46, 54, and/or 74) of the dressing to guide therapeutic gas toward target tissue 22 and/or promote distribution of therapeutic gas within interior volume 78. In this way and others, dressing 18 can be configured to rely less on manifold(s) 46 and/or sorbent layer 54 to guide therapeutic gas toward target tissue 22 and/or distribute therapeutic gas within interior volume 78.

To illustrate, port 94 can be configured to extend through opening 98 of gas-occlusive layer 74. Manifold 46 can include an opening 102 positioned relative to the edges of the manifold such that at least a portion of opening 98 of gas-occlusive layer 74 overlies at least a portion of the opening of the manifold. For example, when port 94 is received by opening 98 of gas-occlusive layer 74, the port can overly at least a portion of opening 102 of manifold 46. Port 94 can be configured to extend through both opening 98 of gas-occlusive layer 74 and through opening 102 of one or more manifolds 46 to guide therapeutic gas toward target tissue 22 and/or distribute therapeutic gas within interior volume 78 (depicted by arrows showing the flow of therapeutic gas). More particularly, as shown in FIG. 4A, port 94 can extend through an upper manifold 46, sorbent layer 54, and a lower manifold 46. As shown in FIG. 4B, port 94 can extend through an upper manifold 46, sorbent layer 54, but not a lower manifold 46 (i.e., the port does not extend through a manifold 46 that is disposed between sorbent layer 54 and target tissue 22). Such a lower manifold 46, as shown in FIG. 4B, can be configured to distribute therapeutic gas from port 94 across the lower manifold (depicted by arrows showing the flow of therapeutic gas).

As shown in FIGS. 4A, 4B, and 5, sorbent layer 54 can include an opening 106 positioned relative to the edges of the sorbent layer such that at least a portion of opening 98 of gas-occlusive layer 74 overlies at least a portion of the opening of the sorbent layer. For example, when port 94 is received by opening 98 of gas-occlusive layer 74 and/or by opening 102 of one or more manifolds 46, the port can overly at least a portion of opening 106 of sorbent layer 54. Port 94 can be configured to extend through opening 98 of gas-occlusive layer 74, through opening 102 of one or more manifolds 46, and opening 106 of sorbent layer 54 to guide therapeutic gas toward target tissue 22 and/or distribute therapeutic gas within interior volume 78.

In this embodiment, dressing 18 comprises a filter 110 configured to filter fluid that flows through opening 98 of gas-occlusive layer 74. For example, filter 110 can be sterile such that the filter provides a viral and/or bacterial barrier. As shown in FIGS. 4A and 4B, filter 110 comprises a layer of material that is bonded to a lower (e.g., tissue-facing) surface of gas-occlusive layer 74. In some embodiments, a filter (e.g., 110) comprises a layer of material that is bonded to an upper surface of a gas-occlusive layer (e.g., 74). Filter 110 can comprise any suitable material, such as, for example, polytetrafluoroethylene (PTFE) (e.g., an expanded PTFE), a polyester, a polyamide, polyolefin, a copolymer thereof, a blend thereof, and/or the like. Filter 110 can have a backing material, such as, for example, a non-woven textile, comprising a polyester, a polyamide, and/or the like. Filter 110 may comprise a hydrophobic material. To illustrate, filter 110 can be configured to allow communication of therapeutic gas into interior volume 78 through opening 98 of gas-occlusive layer and restrict communication of exudate out of the interior volume through the opening of the gas-occlusive layer. Filter 110 can comprise a pore size of approximately 0.05 to 0.15 micrometers (e.g., approximately any one of or between any two of the following: 0.05, 0.07, 0.09, 0.10, 0.11, 0.13, and 0.15 micrometers).

A non-limiting example of filter 110 includes GORE® Microfiltration Media for Medical Devices, which is commercially available from W. L. Gore & Associates, Inc., of Newark, Del., USA.

As shown in FIGS. 1, 2, 4A, and 4B, system 10 can include conduit 19, which is configured to be coupled between therapeutic gas source 14 and dressing 18 to permit fluid communication between the therapeutic gas source and interior volume 78 of the dressing. For example, port 94 can be configured to cooperate with conduit 19 to permit fluid communication between therapeutic gas source 14 and interior volume 78.

Conduit 19 can be configured to be releasably coupled to port 94 and/or to therapeutic gas source 14 (e.g., via a port on the therapeutic gas source having one or more latching and/or interlocking features) such that the therapeutic gas source can be decoupled from dressing 18 without removing the dressing from target tissue 22 and/or tissue 30 surrounding the target tissue.

Referring now to FIG. 7, conduit 19 can include an elongated core 174 comprising a manifold 46b, which is substantially similar to manifold 46 of dressing 18. In this embodiment, a thickness of core 174 is less than 10 percent of a width of the core. Conduit 19 comprises a sheath 178 having a gas-occlusive layer that is similar to gas-occlusive layer 74. Sheath 178 can be disposed around and extend along at least a majority of a length of core 174. Sheath 178 can comprise any suitable material, such as, for example, polyurethane, polyethylene, polyvinyl acetate, polyvinyl chloride, polyvinylidene chloride, isobutylene, a halogenated isomer, a copolymer thereof, or a blend thereof. Sheath 178 may be free of Di(2-ethylhexyl) phthalate (DEHP). In some embodiments, at least a portion of a conduit (e.g., 19) may comprise a tube having a substantially round lateral cross-section (e.g., circular, elliptical, or otherwise round).

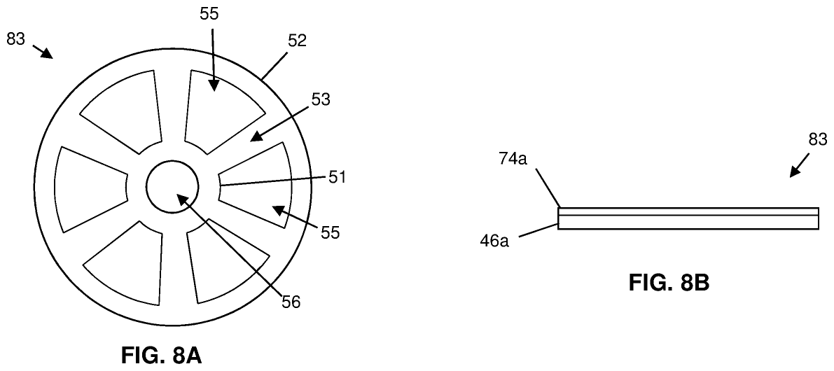

Referring specifically to FIGS. 8A and 8B, shown therein and designated by the reference numeral 83 is a manifolding assembly, suitable for use in some embodiments of the present dressings (e.g., 18, 18a). For example, manifolding assembly 83 may be disposed between sorbent layer 54 and patient-interface layer 26 of dressing 18. Manifolding assembly 83 is configured to facilitate the distribution of therapeutic gas within interior volume 78 and/or to resist protein adhesion and/or clogging due to contact with exudate and target tissue 22.

As shown in FIG. 8A, manifolding assembly 83 comprises an inner ring 51 coupled to an outer ring 52 by one or more channels 53. Channels 53 may be unitary with or bonded to inner ring 51 and/or outer ring 52 (e.g., via high frequency welding, heat staking, and/or an adhesive). As shown, manifolding assembly 83 can comprise a plurality of openings 55 that are defined by channels 53, inner ring 51, and/or outer ring 52. Manifolding assembly 83 is configured such that one or more of plurality of openings 55 permit exudates from target tissue 22 to flow away from the target tissue through the one or more openings. Manifolding assembly 83 can comprise a central opening 56 that is defined by inner ring 51. Central opening 56 may be configured to be aligned with and/or receive port 94 to permit therapeutic gas toward target tissue 22 through the central opening.

Manifolding assembly 83 can comprise a gas occlusive layer 74a, which is substantially similar to gas occlusive layer 74, that is coupled to a manifold 46a, which is substantially similar to manifold 46. Gas occlusive layer 74a and manifold 46a can be bonded to one another via, for example, high frequency welding, heat staking, and/or an adhesive.

Manifolding assembly 83 can be arranged within interior volume 78 such that manifold 46a is closer to target tissue 22 than gas occlusive layer 74a. By disposing gas occlusive layer 74a on an upper surface of manifold 46a, the gas occlusive layer prevents therapeutic gas supplied through central opening 56 from flowing away from target tissue 22 and encourages the flow of therapeutic gas laterally through one or more channels 53 of manifold 46a. Like manifold 46, manifold 46a then permits communication of therapeutic gas to target tissue 22 through the manifold. In this way and others, manifold 46a and gas occlusive layer 74a cooperate to prevent unintended flow of therapeutic gas away from target tissue 22 and encourage the distribution of therapeutic gas through the manifold and interior volume 78.

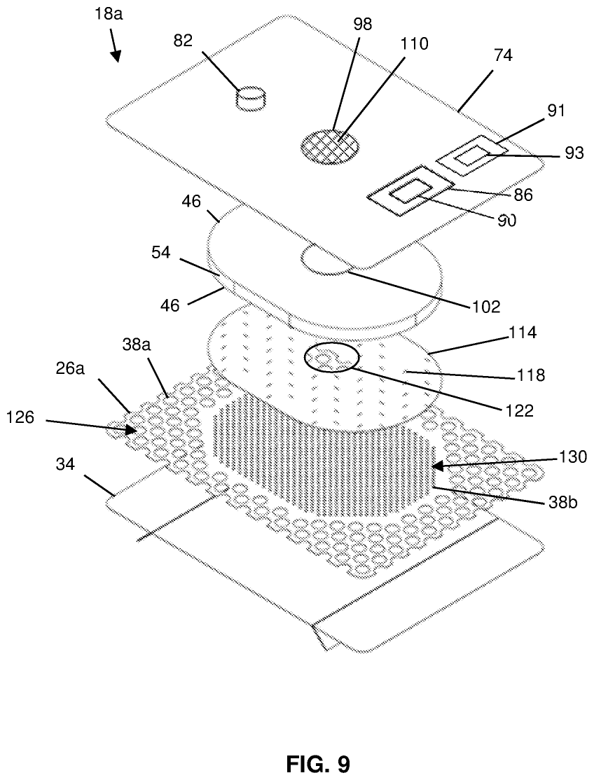

Referring now to FIG. 9, shown therein and designated by the reference numeral 18a is another embodiment of the present wound dressings for facilitating the delivery of therapeutic gas to target tissue 22. Dressing 18a is substantially similar to dressing 18, with the primary exception that dressing 18a comprises a liquid control layer 114 configured to restrict communication of exudate toward the target tissue.

As shown in FIG. 9, liquid control layer 114 can be configured to be disposed below one or more manifolds 46 (e.g., between the manifold(s) and target tissue 22). Sorbent layer 54, and thus, sorbent material 58, can be disposed between one or more manifolds 46 and liquid control layer 114 to capture exudate. In other words, liquid control layer 114 can be configured to be disposed below sorbent layer 54, and thus, below sorbent material 58. In some embodiments, a liquid control layer (e.g., 114) can be disposed between a manifold (e.g., 46) and a sorbent layer (e.g., 54).

Liquid control layer 114 can comprise a plurality of perforations 118 configured to permit exudate to flow away from target tissue 22 through the plurality of perforations and block the flow of exudate toward the target tissue through the plurality of perforations. Each perforation 118 may define an aperture comprising a perimeter that changes (e.g., changes by more than 5 percent) in response to fluid flow through the perforation. Each of perforations 118 of liquid control layer 114 may be substantially equal in size (e.g., as measured by a maximum transverse dimension of the opening), such as, for example, approximately any one of, or between approximately any two of, the following: 1, 2, 3, 4, or 5 millimeters (mm). For example, one or more of plurality of perforations 118 may comprise a slit. One or more perforations 118 can be configured to allow fluid communication through liquid control layer 114 and to prevent gel-blocking in sorbent material 58.

Liquid control layer 114 can comprise any suitable material to restrict communication of exudate toward target tissue 22. For example, liquid control layer 114 can comprise a foam, a non-woven textile, and/or a film. For further example, liquid control layer 114 can comprise a hydrophilic material, such as, for example, a superabsorbent polymer. Liquid control layer 114 can comprise any suitable material, such as, for example, polyethylene. Liquid control layer 114 can comprise a density ranging from 300 to 800 GSM.

Like manifold 46 and sorbent layer 54, liquid control layer 114 can include an opening 122 positioned relative to the edges of the liquid control layer such that at least a portion of opening 98 of gas-occlusive layer 74 overlies at least a portion of the opening of the liquid control layer. For example, when port 94 is received by opening 98 of gas-occlusive layer 74, by opening 102 of manifold 46, and/or by opening 106 of sorbent layer 54, the port can overly at least a portion of opening 122 of liquid control layer 114. Port 94 can be configured to extend through opening 122 of liquid control layer 114 to guide therapeutic gas toward target tissue 22 and/or distribute therapeutic gas within interior volume 78.

Dressing 18a includes a patient-interface layer 26a, which is substantially similar to patient-interface layer 26 with the exception that patient-interface layer 26a comprises a first portion 126 comprising a first plurality openings 38a, each having a first size (e.g., as measured by a maximum transverse dimension of the first opening, examples of which are provided above in relation to openings 38), and a second portion 130 comprising a second plurality of openings 38b, each having a second size (e.g., as measured by a maximum transverse dimension of the second opening) that is at least 50 percent (e.g., 50, 55, 65, 70, 75, 80, 85, 90, or 95 or more percent) smaller than the first size. For example, each of second plurality of openings 38b may be substantially equal in size (e.g., as measured by a maximum transverse dimension of the opening), such as, for example, approximately any one of, or between approximately any two of, the following: 0.1, 0.2, 0.3, 0.4, and 0.5 cm.

In this embodiment, respective ones of second plurality of openings 38b of patient-interface layer 26a and respective ones of plurality of perforations 118 of liquid control layer 114 may be misaligned relative to each other to define a tortuous path for exudate tending to backflow toward target tissue 22, thereby frustrating the backflow of the exudate toward the target tissue. As shown in FIG. 9, patient-interface layer 26a can be configured to be disposed below liquid control layer 114. In some embodiments, a patient-interface layer (e.g., 26a) can be omitted and a liquid control layer (e.g., 114) can be disposed directly onto target tissue (e.g., 22).

Some embodiments of the present methods include coupling one of the present dressings (e.g., 18, 18a) to a patient's tissue (e.g., 22, 30); and introducing therapeutic gas into the interior volume (e.g., 78) of the dressing.

In some embodiments of the present methods, prior to introducing therapeutic gas into the interior volume of the dressing, the method comprises reducing pressure within the interior volume.

In some embodiments of the present methods, introducing therapeutic gas into the interior volume of the dressing is performed at a flow rate that is less than approximately 100 mL/hour, optionally, less than approximately 50 mL/hour. In some embodiments of the present methods, introducing therapeutic gas into the interior volume of the dressing comprises introducing oxygen into the interior volume of the dressing. In some embodiments of the present methods, oxygen introduced into the interior volume of the dressing is produced via electrolysis.

In some embodiments of the present methods, introducing therapeutic gas into the interior volume of the dressing is performed via a conduit (e.g., 19) including: an elongated core (e.g., 174) comprising a foam or a non-woven textile (e.g., 46b); and a sheath (e.g., 178) comprising a gas-occlusive film; wherein the sheath is disposed around and extends along at least a majority of a length of the core. In some embodiments of the present methods, a thickness of the core is less than 10 percent of a width of the core. In some embodiments of the present methods, the sheath comprises polyurethane, polyethylene, polyvinyl acetate, polyvinyl chloride, polyvinylidene chloride, isobutylene, a halogenated isomer, a copolymer thereof, or a blend thereof. In some embodiments of the present methods, the core comprises polyethylene, a polyolefin, a polyether, polyurethane, a co-polyester, a copolymer thereof, or a blend thereof.

### EXAMPLES

The present invention will be described in greater detail by way of specific examples. The following examples are offered for illustrative purposes only and are not intended to limit the invention in any manner. Those of skill in the art will readily recognize a variety of noncritical parameters that can be changed or modified to yield essentially the same results.

### Example 1

### Changes in Oxygen Concentration within a Dressing Comprising the Gas-Occlusive Layer of the Present Disclosure

An SO-220 Fast Response Thermocouple Reference Oxygen Sensor, which is commercially available from Apogee Instruments, Inc., of Logan, Utah, USA, was used to evaluate the oxygen concentration within a first 4-inch by 5-inch dressing and a second 4-inch by 5-inch dressing, each having, in the following order, from farthest to closest to tissue (e.g., 22 or 30): an semi-occlusive film comprising a polyurethane adhesive, a super absorbent textile, a manifold comprising an open cell foam, and a patient interface layer comprising a hydrophilic foam. Each dressing also comprised a cannula inserted through the semi-occlusive film and into the manifold to deliver oxygen to the dressing. The second dressing, in contrast to the first dressing, additionally had a gas-occlusive layer (e.g., 74) comprising a polyurethane film coated with a layer of adhesive at least as thick as the film. The gas-occlusive layer (e.g., 74) of the second dressing was disposed over the semi-occlusive film and sealed around the tissue. Oxygen having a concentration of 99.99 percent was supplied to each of the first and second dressings at a volumetric flow rate of 10 milliliters per hour.

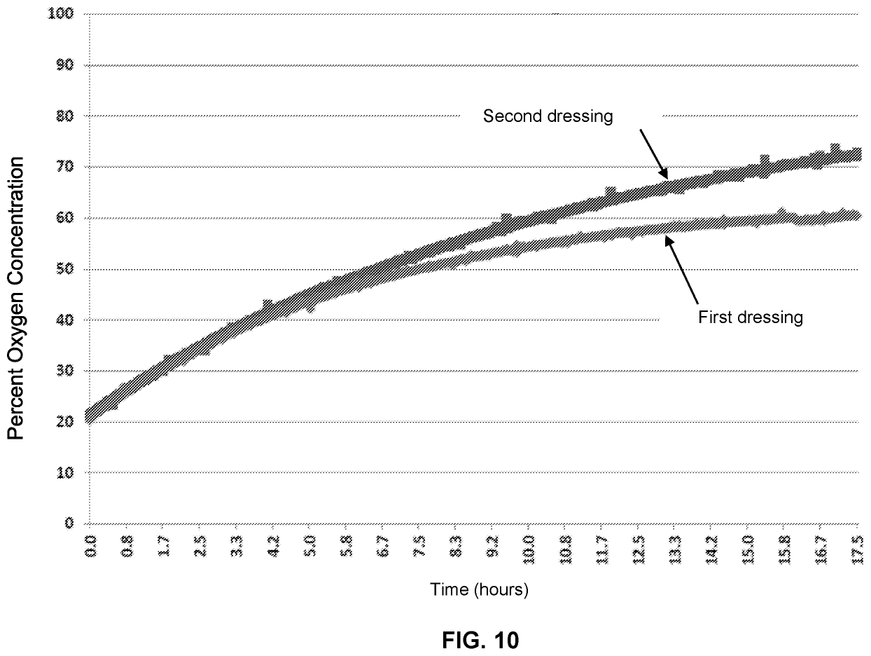

FIG. 10 depicts the resulting oxygen concentration, over time, within an interior volume (e.g., 78) of each of the first and second dressings. As shown in FIG. 10, as compared to the oxygen concentration within the interior volume (e.g., 78) of the first dressing, the oxygen concentration within the interior volume (e.g., 78) of the second dressing reached higher levels in the same amount of time after approximately 3.3 hours. For example, after 17.5 hours, oxygen concentration within the first dressing had reached approximately 60 percent, whereas oxygen concentration within the second dressing had reached approximately 73 percent (i.e., approximately 20 percent more oxygen concentration was present within the second dressing after the same duration of time). This is due, in part, to the addition of the gas-occlusive layer (e.g., 74) on the second dressing, which limits escape of oxygen through the dressing and between the gas-occlusive layer and tissue (e.g., 30) surrounding target tissue (e.g., 22).

### Example 2

### Changes in Oxygen Concentration within a Dressing Comprising the Gas-Occlusive Layer of the Present Disclosure Before and after Liquid Instillation

An SO-220 Fast Response Thermocouple Reference Oxygen Sensor, which is commercially available from Apogee Instruments, Inc., of Logan, Utah, USA, was used to evaluate the levels of oxygen concentration within a 4-inch by 4-inch TIELLE™ Dressing, which is commercially available from Systagenix Wound Management, Limited, of Gargrave, UK (“Systagenix”). The dressing included, in the following order, from farthest to closest to tissue (e.g., 22 or 30): a moisture-permeable polyurethane film with a skin-friendly adhesive, a hydropolymer-based compressed superabsorbent material comprising LIQUALOCK™ Advanced Absorption Technology, which is commercially available from Systagenix, a manifolding assembly (e.g., 83) having a gas occlusive layer (e.g., 74a) adhered to a porous polyethylene manifold (e.g., 46a), the manifolding assembly also having an inner ring (e.g., 51) coupled to an outer ring (e.g., 52) by six channels (e.g., 53), and a silicone-based patient-interface layer (e.g., 26).

The dressing was modified to additionally include a gas-occlusive layer (e.g., 74) comprising a polyurethane film with high application of adhesive that was disposed over the moisture-permeable polyurethane film and sealed around the tissue. Oxygen having a concentration of 99.99 percent was supplied to the dressing at a volumetric flow rate of 15 milliliters per hour using a NATROX® Oxygen Generator, which is commercially available from Inotec AMD Ltd., of Cambridge, England.

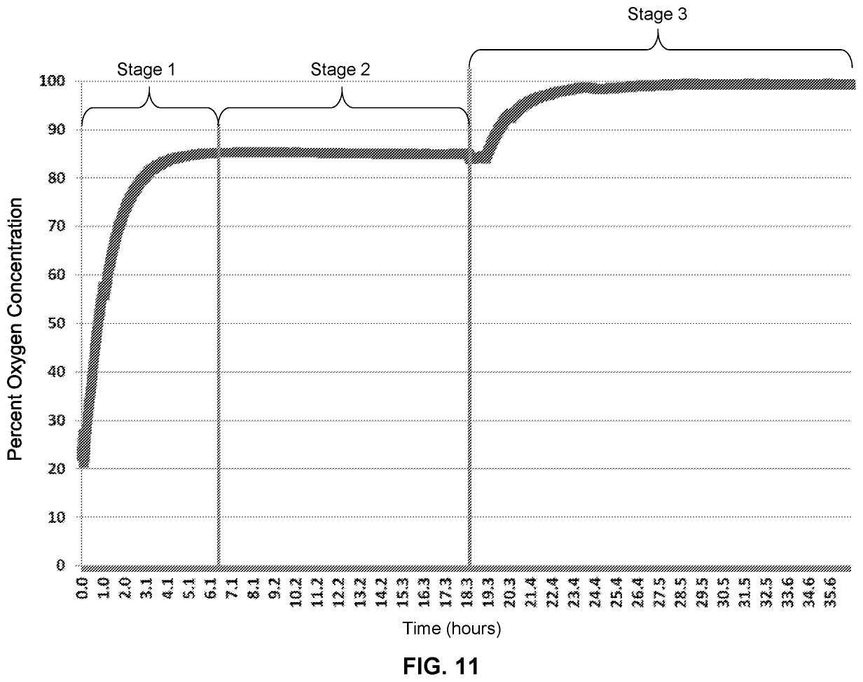

FIG. 11 depicts the resulting oxygen concentration, over time, within an interior volume (e.g., 78) of the dressing. As shown, the oxygen concentration data was divided into three stages: Stage 1, which lasted from the 0.0 hour mark to the 6.5 hour mark; Stage 2, which lasted from the 6.5 hour mark to the 18.5 hour mark, and Stage 3, which lasted from the 18.5 hour mark to the 35.6 hour mark. As shown in FIG. 11, the internal volume (e.g., 78) of the dressing reached an oxygen concentration of approximately 85 percent after supplying oxygen for approximately 6.5 hours. During Stage 1, liquid, which simulated exudate, was not introduced into the interior volume (e.g., 78).

During Stage 2, the dressing maintained approximately 85 percent oxygen concentration without interruption, as representative of a simulated topical oxygen therapy. At the beginning of Stage 3 (i.e., at approximately the 18.5 hour mark), protein liquid with red food coloring was instilled—a total of 40 mL at a rate of 20 mL/hour—into the dressing to simulate the absorption of exudate into the dressing. It was observed, by the color change and by the swelling of the superabsorbent material of the dressing, that the volume of the interior volume (e.g., 78) had been filled by the superabsorbent material, thereby further improving the occlusiveness of the dressing at least because the superabsorbent material provided an additional barrier for liquid flow out of the dressing. Due at least in part to the superabsorbent material's filling the interior volume (e.g., 78) and the subsequent increased occlusiveness of the dressing, the dressing exhibited an increase in the oxygen concentration within the interior volume to approximately 99 percent in less than six hours after the start of fluid instillation (i.e., less than six hours after the 18.5 hour mark). Further, as shown, after the instillation of protein liquid, the oxygen concentration within the dressing increased at an even faster rate until it peaked just under 99 percent.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1.-89. (canceled)

90. A dressing configured to be coupled to tissue to facilitate delivery of therapeutic gas to the tissue, the dressing comprising:
a manifold that defines a plurality of gas passageways, the manifold configured to allow communication of therapeutic gas to the tissue;
a sorbent layer including a foam or a non-woven textile, the sorbent layer configured to be disposed below or above the manifold and to draw exudate away from the tissue; and
a gas-occlusive layer configured to be disposed over the manifold and coupled to the tissue such that an interior volume containing the manifold is defined between the gas-occlusive layer and the tissue and the gas-occlusive layer limits escape of therapeutic gas from the interior volume.

91. The dressing of claim 90, wherein the sorbent layer has a plurality of perforations and/or openings configured to allow communication of therapeutic gas to the tissue.

92. The dressing of claim 90, wherein the sorbent layer comprises a superabsorbent polymer.

93. The dressing of claim 90, wherein the sorbent layer comprises a carbon filter.

94. The dressing of claim 90, comprising a patient-interface layer configured to be disposed below the sorbent layer and in contact with the tissue, the patient-interface layer defining a plurality of openings configured to allow communication of therapeutic gas and exudate through the patient-interface layer.

95. The dressing of claim 94, wherein the patient-interface layer comprises a polymer, optionally, silicone, polyethylene, ethylene vinyl acetate, a copolymer thereof, or a blend thereof.

96. The dressing of claim 90, comprising a liquid control layer having a plurality of perforations, the liquid control layer configured to be disposed below the manifold to restrict communication of exudate toward the tissue.

97. The dressing of claim 96, wherein the sorbent layer is configured to be disposed between the manifold and the liquid control layer to capture exudate.

98. The dressing of claim 96, wherein the liquid control layer comprises a hydrophilic material, optionally, a superabsorbent material.

99. The dressing of claim 90, wherein the manifold comprises polyethylene, a polyolefin, a polyether, polyurethane, a co-polyester, a copolymer thereof, or a blend thereof.

100. The dressing of claim 90, comprising a second manifold defining a plurality of gas passageways and configured to allow communication of therapeutic gas to the tissue, wherein the sorbent layer is configured to be disposed between the manifold and the second manifold.

101. The dressing of claim 90, comprising:
one or more ports coupled to or defined by the gas-occlusive layer;
wherein the one or more ports are configured to permit communication of therapeutic gas through the gas-occlusive layer and into the interior volume.

102. The dressing of claim 101, comprising, for at least one of the one or more ports, a filter coupled to the gas-occlusive layer and configured to filter fluid that flows through the port.

103. The dressing of claim 102, wherein the filter comprises polytetrafluoroethylene, a polyester, a polyamide, a copolymer thereof, or a blend thereof.

104. The dressing of claim 90, comprising a valve coupled to the gas-occlusive layer and configured to relieve pressure within the interior volume when pressure within the interior volume exceeds a threshold pressure.

105. The dressing of claim 90, comprising a sensor configured to detect a presence of therapeutic gas within the interior volume.

106. A system comprising:
a dressing having
a manifold that defines a plurality of gas passageways, the manifold configured to allow communication of therapeutic gas to the tissue;
a sorbent layer including a foam or a non-woven textile, the sorbent layer configured to be disposed below or above the manifold and to draw exudate away from the tissue;
a gas-occlusive layer configured to be disposed over the manifold and coupled to the tissue such that an interior volume containing the manifold is defined between the gas-occlusive layer and the tissue and the gas-occlusive layer limits escape of therapeutic gas from the interior volume;
an oxygen source; and
a conduit configured to be coupled between the oxygen source and the dressing to permit communication of oxygen from the oxygen source into the interior volume of the dressing.

107. The system of claim 106, wherein the oxygen source comprises an electrolytic oxygen source.

108. The system of claim 106, wherein the oxygen source is configured to provide oxygen at a flow rate that is less than approximately 100 milliliters per hour (mL/hour).

109. The system of claim 107, wherein the conduit includes:
an elongated core comprising a foam or a non-woven textile; and
a sheath comprising a gas-occlusive film;
wherein the sheath is disposed around and extends along at least a majority of a length of the core.

110. A method comprising:
coupling a dressing to a patient's tissue, wherein the dressing comprises:
a manifold that defines a plurality of gas passageways, the manifold configured to allow communication of therapeutic gas to the tissue;
a sorbent layer including a foam or a non-woven textile, the sorbent layer configured to be disposed below or above the manifold and to draw exudate away from the tissue;
a gas-occlusive layer configured to be disposed over the manifold and coupled to the tissue such that an interior volume containing the manifold is defined between the gas-occlusive layer and the tissue and the gas-occlusive layer limits escape of therapeutic gas from the interior volume; and
introducing therapeutic gas into the interior volume of the dressing.

111. The method of claim 110, wherein introducing therapeutic gas into the interior volume of the dressing is performed at a flow rate that is less than approximately 100 mL/hour, optionally, less than approximately 50 mL/hour.

112. The method of claim 110, wherein introducing therapeutic gas into the interior volume of the dressing comprises introducing oxygen into the interior volume of the dressing.

113. The method of claim 112, wherein oxygen introduced into the interior volume of the dressing is produced via electrolysis.

114. The method of claim 110, comprising, prior to introducing therapeutic gas into the interior volume of the dressing, reducing pressure within the interior volume.
